(19) United States
(12) Patent Application Publication
(10) Pub. No.: US 2019/0374752 A1
(43) Pub. Date: Dec. 12, 2019

(54) ADHESIVE DEVICE THAT ANCHORS SURGICALLY PLACED DRAIN TUBES TO SKIN

(71) Applicant: Mohammad Hossein Niayesh, Detroit, ME (US)

(72) Inventor: Mohammad Hossein Niayesh, Detroit, ME (US)

(21) Appl. No.: 16/002,149

(22) Filed: Jun. 7, 2018

(51) Int. Cl. A61M 27/00

(52) U.S. Cl. CPC A61M 27/00; A61M 2209/088

(57) ABSTRACT

A medical device that anchors surgically placed drain tubes to a patient is provided. The medical device has a conical shaped body defining a broad base tapering into a centrally disposed spout, wherein a passageway extends through both the spout and the base. The base terminates at a contact surface having an adhesive element, as does the inner circumference of the passageway. The body may be separated along an interface so that the body is movable between a separate and a closed condition for inserting and securing the surgical drain tube in the passageway, respectively. The contact surface adhesive element anchors the medical device, and thus the drain tube, to the patient.

### BACKGROUND OF THE INVENTION

The present invention relates to medical devices and, more particularly, to a medical device that anchors surgically placed drain tubes to the patient.

Securing surgical drain tubes to the skin is difficult and as a result many accidentally fall out, needing reinsertion at additional cost and trauma. Traditionally surgical drain tubes are stitched to the skin and then held in place by bulky adhesive tape. Such tape tends to loosen, allowing in the surgical drain tubes to become unmoored, fall out or be accidentally displaced. The adhesive tapes are designed for a flat surface and have not been designed to secure a drain tube coming out of the skin at a perpendicular angle. There is at present no purpose built surgical drain tube anchor that is easy to apply, effective, and not unsightly.

As can be seen, there is a need for an anchoring device that anchors surgically placed drain tubes. The present invention embodies a medical device providing a broad adhesive base for securing to the patient's skin, the device also includes an integral central spout providing an adhesive passageway that communicates through the adhesive base. The broad adhesive base anchors to the patient's skin so that a surgically placed tube drains can be safely and securely received through the adhesive passageway, eliminating the need for bulky, unsightly, and ineffective dressings used to traditionally secure them in place

### SUMMARY OF THE INVENTION

In one aspect of the present invention, a medical device for anchoring surgical drain tubes to a patient includes a base tapering inward from a contact surface toward a centrally disposed spout; a passageway extending through said base and spout; a base adhesive disposed along the contact surface; and a passageway adhesive disposed along an inner circumference of the passageway.

In another aspect of the present invention, the medical device for anchoring surgical drain tubes to a patient includes a base tapering inward from a contact surface toward a centrally disposed spout; a passageway extending through said base and spout; a base adhesive disposed along the contact surface; a base peel-away layer engaging the base adhesive; a passageway adhesive disposed along an inner circumference of the passageway; a passageway peel-away layer engaging the passageway adhesive; a longitudinal slit extending along the device from the contact surface to the passageway so that the device is movable between a separated condition and the closed condition; and the device comprises a material biasing the device in the closed condition.

In yet another aspect of the present invention, a method for securing a surgical drain tube to a patient, comprising the steps of: providing the above-mentioned medical device; urging the device to the separated condition; placing the surgical drain tube in the passageway; moving the device to the closed condition so that the passageway adhesive engages an outer circumference of the surgical drain tube; and adhering the base adhesive to the patient.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a perspective view of an exemplary embodiment of the present invention, shown in use;

FIG. 3 is a cross-sectional view of an exemplary embodiment of the present invention, taken along line 3-3 in FIG. 1; and

FIG. 4 is a cross-sectional view of an exemplary embodiment of the present invention, taken along line 4-4 in FIG. 2.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a medical device that anchors surgically placed drain tubes to a patient. The medical device has a conical shaped body defining a broad base tapering into a centrally disposed spout, wherein a passageway extends through both the spout and the base. The base terminates at a contact surface having an adhesive element, as does the inner circumference of the passageway. The body may be separated along an interface so that the body is movable between a separate and a closed condition for inserting and securing the surgical drain tube in the passageway, respectively. The contact surface adhesive element anchors the medical device, and thus the drain tube, to the patient.

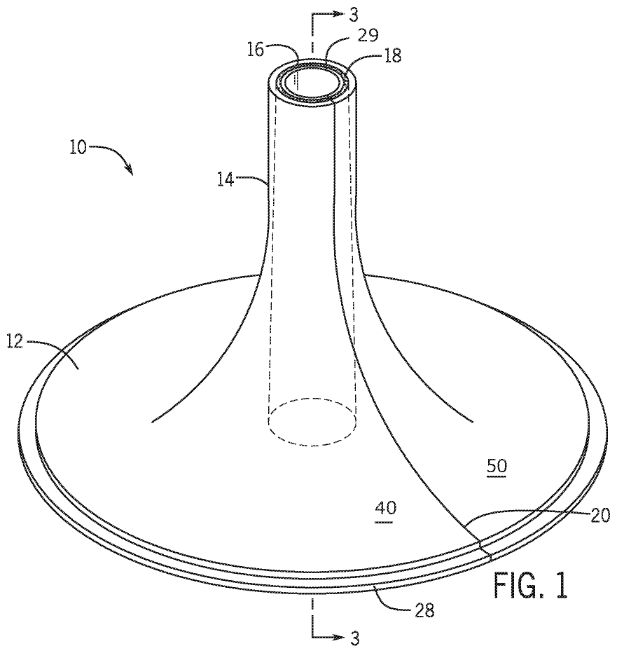

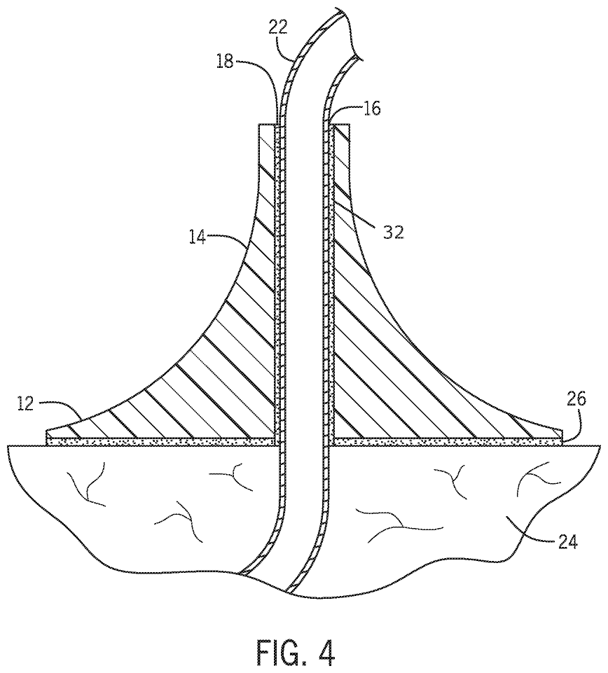

Referring to FIGS. 1 through 4, the present invention may include a medical device 10 having a circular base 12 defining a generally planar contact surface 30. An adhesive 26 may be disposed along said contact surface 30. The base adhesive 26 may be a coating, tape, or the like. Prior to use, the base adhesive 26 may be engaged by a removable base peel-away layer 28.

Extending, generally perpendicular, from a center of the base 12 may be a spout 14 defining a passageway 16. Along the circumferential surface 32 of the passageway 16 may be passageway adhesive 18. The passageway adhesive 18 may be a coating, tape, or the like. Prior to use, the passageway adhesive 18 may be engaged by a removable passageway peel-away layer 29.

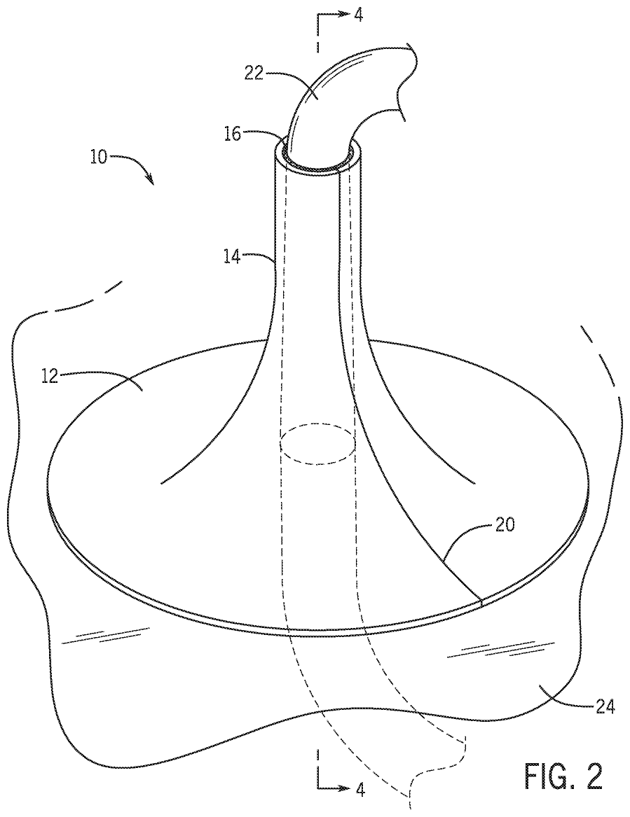

Referring to FIG. 2, the base 12 and its integral spout 14 provide a slit 20 from a periphery of the base 12, extending to the centrally disposed spout 14, along a longitudinal length of the spout 14 to a distal end thereof, and then turning to an opening communicating to the passageway 16. The slit 20 divides the medical device 10 into two separable portions, a first portion 40 and a second portion 50. A user can thus urge the first and second portions 40 and 50 away from each other creating a void therebetween, which in turn expands the size of the passageway 16 in such a separated condition; the first and second portions 40 and 50 movable between the separated and closed conditions. The base 12 and integral spout 14 may be made of a material that biases the medical device 10 in the closed condition, such as gel or plasticized materials. In the separated condition objects dimensioned to be received through the passageway 16, such as a surgical drain tube 22, can be more easily received through the passageway 16. When moved back to the closed conditions, the passageway 16 abuts against the surgical drain tube 22. Also, in the separated condition the passageway peel-away layer 29 covering the passageway adhesive 18 may be more easily peeled off because of the expanded dimensionality. Moving the medical device 10 to the closed condition, the surgical drain tube 22 sticks to the exposed passageway adhesive 18. Then the user may remove the base peel-away layer 28 and adhere to the contact surface 30 to the flesh 24 of the patient, as a result holding the surgical drain tube 22 and anchoring it.

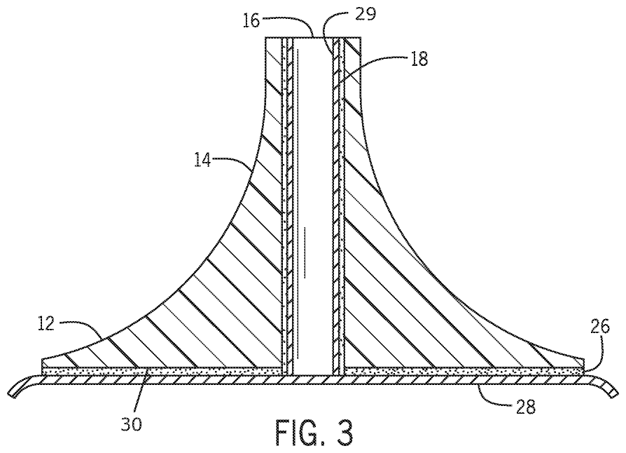

The medical device 10, the base 12 and spout 14 defining the through passageway 16, has a generally conic shape as illustrated in FIG. 3. When the passageway operatively associates with the drain tube 22, bending forces applied to the drain tube 22 will distributed through the above-mentioned conic shape, thereby ameliorating such forces and lessening the likelihood such forces result in the drain tube 22 becomes unmoored to the location where the contact surface 30 as secured them too. Likewise, the breadth of the contact surface 30 relative to the spout 14 counters related tipping forces.

There are a variety of sizes of drain tubes (such as small abdominal tubes, large abdominal tubes, chest tubes, and the like) that the present invention may be applicable for, and as a result the dimensions of the structural components may vary. The thickness of the medical device 10 may be 0.5 millimeters (mm), 0.8 mm, 1.0 mm or the like. The diameter of the circular base 12 may be 4 centimeters (cm), 9 cm, 15 cm or the like. The spout 14/passageway 16 height and inner diameter may be 2 cm and 3 mm, 4 cm and 6 mm, 6 cm and 9 mm, and the like.

A method of using the present invention may include the following. The medical device 10 disclosed above may be provided. A user may place the surgical drain tube 22 at the slit 20, and in the separated condition move the base 12 until the surgical drain tube 22 comes to rest in the passageway 16. Then the user would peel the passageway peel-away layer 29 off the passageway 16 and squeeze the passageway adhesive 18 to surgical drain tube 22. Then the user may peel the base peel-away layer 28 off the contact surface 30 and secure the contact surface to the body 24 of the patient at the drain exit site.

It should be understood, of course, that the foregoing relates to exemplary embodiments of the invention and that modifications may be made without departing from the spirit and scope of the invention as set forth in the following claims.

What is claimed is:

1. A device for anchoring surgical drain tubes to a patient, comprising:
a base tapering inward from a contact surface toward a centrally disposed spout;
a passageway extending through said base and spout;
a base adhesive disposed along the contact surface; and
a passageway adhesive disposed along an inner circumference of the passageway.

2. The device of claim 1, further comprising a longitudinal slit extending along the device from the contact surface to the passageway so that the device is movable between a separated condition and the closed condition.

3. The device of claim 2, wherein the longitudinal slit takes the shortest route from the contact surface to the passageway.

4. The device of claim 1, further comprising a passageway peel-away layer engaging the passageway adhesive.

5. The device of claim 1, further comprising a base peel-away layer engaging the base adhesive.

6. The device of claim 2, wherein the device comprises a material biasing the device in the closed condition.

7. The device of claim 1, comprising:
the base having a diameter between four cm to 13.5 cm;
the passageway having a height between two cm and six cm; and
the passageway having a uniform inner diameter between three mm and nine mm.

8. A device for anchoring surgical drain tubes to a patient, comprising:
a base tapering inward from a contact surface toward a centrally disposed spout;
a passageway extending through said base and spout;
a base adhesive disposed along the contact surface;
a base peel-away layer engaging the base adhesive;
a passageway adhesive disposed along an inner circumference of the passageway;
a passageway peel-away layer engaging the passageway adhesive;
a longitudinal slit extending along the device from the contact surface to the passageway so that the device is movable between a separated condition and the closed condition; and
the device comprises a material biasing the device in the closed condition.

9. The device of claim 8, comprising:
the base having a diameter between four cm to 13.5 cm;
the passageway having a height between two cm and six cm; and
the passageway having a uniform inner diameter between three mm and nine mm.

10. A method for securing a surgical drain tube to a patient, comprising the steps of:
providing the device of claim 2;
urging the device to the separated condition;
placing the surgical drain tube in the passageway;
moving the device to the closed condition so that the passageway adhesive engages an outer circumference of the surgical drain tube; and
adhering the base adhesive to the patient.
